(19) United States
(12) Patent Application Publication
(10) Pub. No.: US 2013/0325129 A1
(43) Pub. Date: Dec. 5, 2013

(54) TEXTURED IMPLANT DEVICE

(71) Applicant: OSSAWARE BIOTECH CO., LTD., Changhua Hsien (TW)

(72) Inventor: Meng Feng HUANG, Changhua Hsien (TW)

(73) Assignee: Ossaware Biotech Co., Ltd., Changhua Hsien (TW)

(21) Appl. No.: 13/847,608

(22) Filed: Mar. 20, 2013

(30) Foreign Application Priority Data
May 30, 2012 (TW) 101119426

(51) Int. Cl. A61F 2/44

(52) U.S. Cl. CPC A61F 2/44
USPC 623/17.16

(57) ABSTRACT

A textured bone graft or implant device includes a spatial member having a number of three dimensional units coupled together, the three dimensional units each include a number of bars coupled together to form a number of openings and to form a porous structure for the spatial member and for allowing the bone materials of the user to be grown and filled into the openings of the textured implant device, the spatial member includes a chamber for engaging with a casing, and the casing includes a compartment for engaging with an inner core, the casing and the inner core each include a number of bars for forming a resilient or flexible or compressible or deformable structure.

### BACKGROUND OF THE INVENTION

1. Field of the Invention

The present invention relates to a textured bone graft or implant device, and more particularly to a textured bone graft or implant device including a porous or perforated structure having a number of orifices or cavities or openings formed therein and defined by a number of cubical or parallelepiped or spatial units for increasing the resilience of the textured bone graft or implant device and for preventing the bone materials or members of the user from being damaged by the textured implant device and for allowing the bone materials of the user to be grown and filled into the orifices or cavities or openings of the textured bone graft or implant device.

2. Description of the Prior Art

Typical bone grafts, intervertebral implants, intervertebral spacers, textured bone graft devices or the like have been developed and provided for being filled or fitted or engaged into the bone materials or members of the human or user for repairing bone defects, such as the damaged or degenerated spinal disc in a spinal column or vertebrae of a human or user, or the like, and comprise a constructed or textured spatial member for engaging into the bone materials or members of the user and for repairing bone defects.

For example, U.S. Pat. No. 5,865,848 to Baker, U.S. Pat. No. 5,888,227 to Cottle, U.S. Pat. No. 5,989,289 to Coates et al., U.S. Pat. No. 6,139,579 to Steffee et al., and U.S. Pat. No. 6,511,509 to Ford et al. disclose several of the typical bone grafts, intervertebral implants, intervertebral spacers, textured bone graft devices or the like and each also comprising a constructed or textured spatial member, such as an artificial spinal disc prosthesis, or the like for fitting or engaging into the bone materials or members, such as the damaged or degenerated spinal column or vertebrae or spinal disc of the human or user.

However, the conventional or typical bone grafts, intervertebral implants, intervertebral spacers, textured bone graft devices or the like comprise a constructed or textured spatial member that includes a solid and stable structure and that may include a great hardness that the bone materials or members of the user may be worn out or damaged or degenerated by the conventional or typical bone grafts, intervertebral implants, intervertebral spacers, textured bone graft devices or the like after use. In addition, the conventional bone grafts, intervertebral implants, intervertebral spacers, textured bone graft devices or the like comprise a solid structure that the bone materials of the human or user may not be easily grown and filled into the conventional or typical bone grafts, intervertebral implants, intervertebral spacers, textured bone graft devices or the like, such that the conventional or typical bone grafts, intervertebral implants, intervertebral spacers, textured bone graft devices or the like may not be formed integral with the bone materials or members of the user.

The present invention has arisen to mitigate and/or obviate the afore-described disadvantages of the conventional textured bone graft or implant devices.

### SUMMARY OF THE INVENTION

The primary objective of the present invention is to provide a textured bone graft or implant device including a porous or perforated structure having a number of orifices or cavities or openings formed therein and defined by a number of cubical or parallelepiped or spatial units for increasing the resilience of the textured bone graft or implant device and for preventing the bone materials or members of the user from being damaged by the textured bone graft or implant device and for allowing the bone materials of the user to be grown and filled into the orifices or cavities or openings of the textured bone graft or implant device.

In accordance with one aspect of the invention, there is provided a textured bone graft or implant device comprising a spatial body member including a number of three dimensional units coupled together to form a spatial and three dimensional structure for the spatial body member, the three dimensional units each including a number of bars coupled together to form a number of openings therein and for forming a porous, resilient or flexible or compressible or deformable structure for the spatial body member and for allowing the bone materials of the user to be grown and filled into the openings of the spatial body member of the textured implant device.

The spatial body member includes a chamber formed therein, and a casing is engaged into the chamber of the body member and secured or coupled to the body member. The casing includes a number of three dimensional units coupled together to form a spatial and three dimensional and resilient or flexible or compressible or deformable structure for the casing.

The three dimensional units of the casing each include a number of bars coupled together to form a number of openings and to form a porous structure for the casing. The casing includes a compartment formed therein, and an inner core engaged into the compartment of the casing and secured or coupled to the casing.

The inner core includes a number of three dimensional units coupled together to form a spatial and three dimensional structure for the inner core. The three dimensional units of the inner core each include a number of bars coupled together to form a number of spaces therein and to form a porous structure for the inner core. The inner core includes at least one insert engaged therein which includes a solid structure.

A receptacle may be engaged onto the outer peripheral portion of the body member. The receptacle includes at least one flap extended outwardly therefrom for coupling to the bone materials. The receptacle includes a number of apertures formed therein for allowing the bone materials of the user to be grown and filled into the orifices or cavities or openings of the textured implant device.

The spatial body member is made of a material selected form stainless steel, titanium, gold, silver, stellite, oxidized cobalt, tantalum, polyetherether ketone (PEEK), polyetherketoneketone (PEKK), ultra-high-molecular-weightpolyethylene (UHMWPE), polylactide (PLA), polyglycolicacid (PGA), or polylacticcoglycolicacid (PLGA).

In accordance with another aspect of the invention, there is provided a textured bone graft or implant device comprising a spatial body member including a chamber formed therein and formed and defined by two side wall panels, a rear wall panel, and a front wall panel, and a frame device including an upper frame member attached onto top of the spatial body member, and a lower frame member attached onto bottom of the spatial body member, the upper and the lower frame members including a front portion secured together.

The upper and the lower frame members of the frame device each include an inner peripheral recess formed therein for engaging with the spatial body member. The upper and the lower frame members of the frame device each include a rectangular shape.

The spatial body member includes a plurality of peripheral ropes engaged in the chamber of the spatial body member and attached to the side wall panels and the rear wall panel and the front wall panel of the spatial body member.

The spatial body member includes a plurality of three dimensional units coupled together to form a spatial and three dimensional structure for the spatial body member, the three dimensional units each include a plurality of bars coupled together to form a plurality of openings therein and for forming a porous structure for the spatial body member.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a partial perspective view of a textured bone graft or implant device in accordance with the present invention, for fitting or engaging into the bone materials or members of the user;

FIG. 2 is an enlarged partial perspective view of the textured bone graft or implant device, in which a portion of the textured bone graft or implant device has been cut off for showing the inner structure of the textured bone graft or implant device;

FIG. 3 is a cross sectional view of the textured implant device, taken along lines 3-3 of FIG. 2;

FIG. 4 is a partial cross sectional view illustrating a portion of the textured implant device;

FIG. 5 is another partial cross sectional view illustrating a prototype or material for forming the portion of the textured implant device;

FIG. 6 is a partial perspective view of the prototype or material for forming the portion of the textured implant device;

FIG. 7 is a partial side plan schematic view illustrating the operation of the textured implant device;

FIG. 8 is a partial perspective view illustrating a cubical or parallelepiped or spatial unit of the prototype or material for forming the portion of the textured implant device;

FIGS. 9, 10, 11, 12 are partial perspective views similar to FIG. 7, illustrating the other arrangement of the cubical or parallelepiped or spatial unit of the prototype or material for forming the portion of the textured implant device;

FIGS. 13, 14, 15 are partial cross sectional views similar to FIG. 4, illustrating the other arrangements of the cubical or parallelepiped or spatial unit of the prototype or material for forming the portion of the textured implant device;

FIG. 16 is a partial perspective view illustrating the further arrangement of the prototype or material of the textured implant device;

FIG. 17 is a partial cross sectional view illustrating the still further arrangement of the prototype or material of the textured implant device;

FIG. 18 is a side plan schematic view illustrating the still further arrangement of the prototype or material of the textured implant device;

FIGS. 19, 20, 21, 22 are side plan schematic views illustrating the still further arrangement of the prototype or material of the textured implant device;

FIG. 23 is another partial side plan schematic view similar to FIG. 7, illustrating the operation of a still further arrangement of the textured implant device;

FIG. 24 is a perspective view of the textured implant device as shown in FIG. 23;

FIG. 25 is another perspective view similar to FIG. 24, in which one half or a portion of the textured implant device has been cut off for showing the inner structure of the textured implant device;

FIG. 26 is an enlarged partial perspective view of the textured implant device as shown in FIGS. 23-25;

FIG. 27 is a cross sectional view of the textured implant device, taken along lines 27-27 of FIG. 24; and

FIG. 28 is another cross sectional view similar to FIG. 27, illustrating the operation of the textured implant device as shown in FIGS. 23-27.

### DETAILED DESCRIPTION OF THE PREFERRED EMBODIMENT

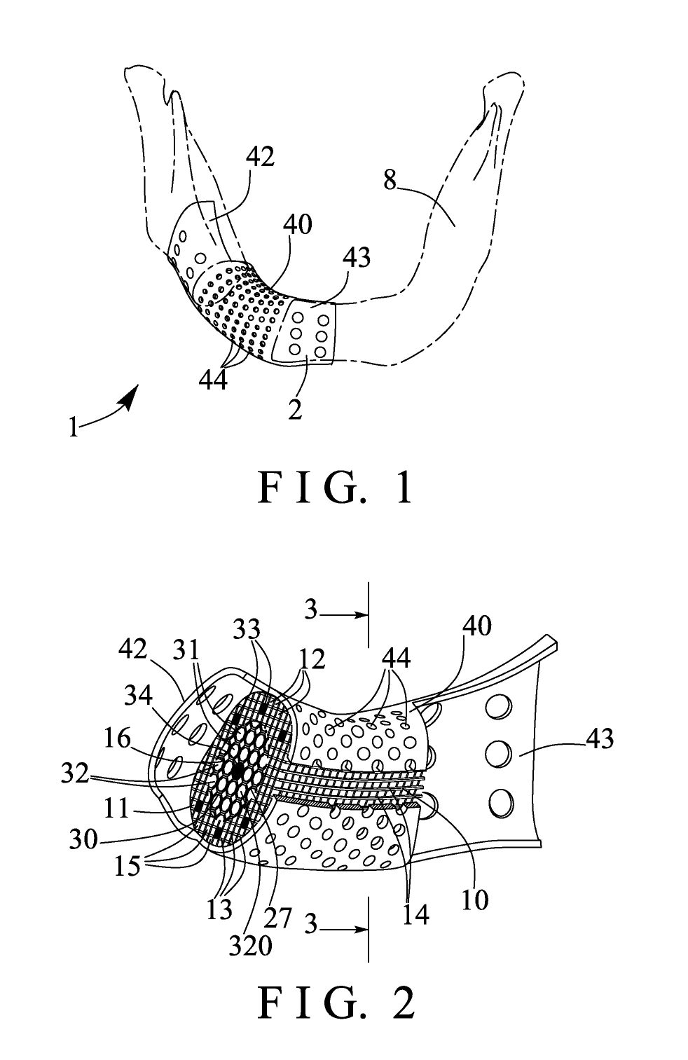

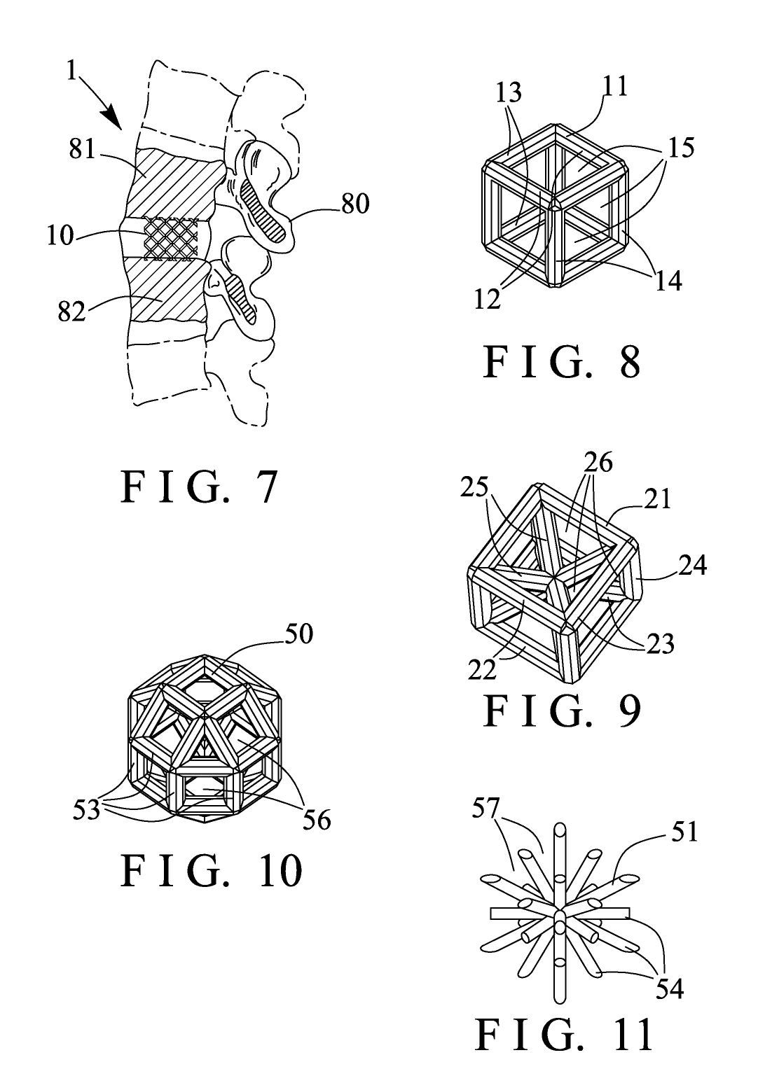

Referring to the drawings, and initially to FIGS. 1-3, a textured bone graft or implant device 1 in accordance with the present invention is generally provided for being filled or fitted or engaged into the damaged or degenerated bone materials or members 8, 80, such as the mandible 8 (FIG. 1), the spinal column 80 or vertebrae of a human or user (FIG. 7), or the like for repairing bone defects, for example, the textured implant device 1 may be directly engaged into the damaged or degenerated portion of the mandible 8 (FIG. 1), or may be disposed or attached or mounted or secured or engaged into the space between two vertebral bodies 81, 82 (FIG. 7) for forming or acting as the intervertebral disk and for sustain the vertebral bodies 81, 82 in place.

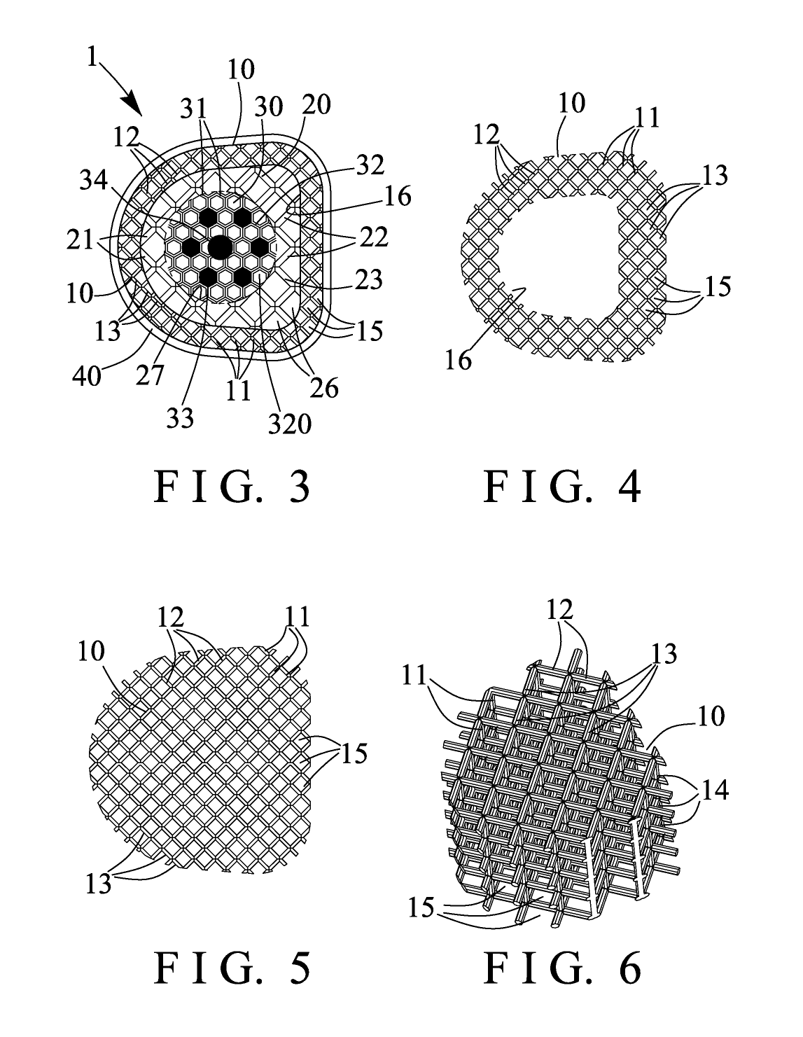

For example, the textured implant device 1 comprises a constructed or textured three dimensional or spatial body member 10 (FIGS. 3, 4) including a porous or perforated structure formed and defined by a number of cubical or parallelepiped or three dimensional or spatial units 11 (FIG. 8) each of which include a number of longitudinal bars 12, a number of lateral bars 13, and a number of vertical bars 14 (FIGS. 2, 6) for forming the cubical or parallelepiped or three dimensional or spatial structure selected from a triangle, rectangle, pentagon, hexagonal, octangle, and the like, and for forming or defining a number of orifices or cavities or openings 15 in the spatial units 11 and/or in the spatial body member 10, and the longitudinal bars 12 and the lateral bars 13 and the vertical bars 14 of the spatial units 11 may be molded or welded or adhered or secured or coupled together in regular to form the three dimensional or spatial body member 10.

As shown in FIGS. 3-4, 13 and 15, illustrated is a material or prototype 100 having a cubical or parallelepiped or three dimensional or spatial structure and to be cut or bored or machined or drilled or milled to form a chamber 16 in the prototype 100 and to be cut or machined or milled to form the cubical or parallelepiped or three dimensional or spatial structure for the prototype 100 that includes a selected or predetermined shape or contour for forming the three dimensional or spatial body member 10. The textured implant device 1 further includes a frame or casing 20 disposed or engaged into the chamber 16 of the body member 10 (FIGS. 3, 14) and molded or welded or adhered or secured or coupled to the body member 10, and the casing 20 also includes a porous or perforated structure formed and defined by a number of cubical or parallelepiped or three dimensional or spatial units 21.

It is preferable, but not necessary that the spatial units 21 are identical in shape, contour, configuration, structure or the like to the spatial units 11, and/or may include a compactness or density identical to or different from that of the spatial units 11. For example, the spatial units 21 may include a compactness or density less than that of the spatial units 11 (FIGS. 3, 14), and as shown in FIG. 9, the spatial units 21 may also include a number of longitudinal bars 22, a number of lateral bars 23, a number of vertical bars 24, and a number of inclined or tilted or additional bars 25 engaged with or molded or welded or adhered or secured or coupled to the bars 22, 23, 24 for forming the cubical or parallelepiped or three dimensional or spatial structure the spatial units 21, and also for forming or defining a number of orifices or cavities or openings 26 in the spatial units 21.

The casing 20 may also be cut or bored or machined or drilled or milled to form a compartment 27 in the casing 20 (FIG. 3) for receiving or engaging with an inner core 30. The inner core 30 also includes a porous or perforated structure formed and defined by a number of cubical or parallelepiped or three dimensional or spatial units 31. It is also preferable, but not necessary that the spatial units 31 are identical in shape, contour, configuration, structure or the like to the spatial units 11, 21, and/or may include a compactness or density identical to or different from that of the spatial units 11, 21. For example, the spatial units 31 may include a shape, contour, configuration, structure or the like different from that of the spatial units 11, 21 and also formed or shaped by a number of bars 32 and/or a number of holes or spaces 320, and may include a hexagonal shape (FIGS. 2, 3), and may include one or more inserts 33, 34 disposed or engaged therein, in which the inserts 33, 34 may include a solid configuration or structure.

The textured implant device 1 further includes an outer frame or sheath or peripheral wall or housing or receptacle 40 (FIGS. 1-3, 15-16) attached or mounted or secured or engaged or molded or welded or adhered or secured or coupled onto the outer peripheral portion of the body member 10, or may include a receptacle 41 (FIG. 18) having a shape, contour, configuration, structure or the like different from that of the receptacle 40. As shown in FIGS. 1 and 2, the receptacle 40 may further include one or more (such as two) ears or panels or flaps 42, 43 extended outwardly therefrom for solidly and stably attaching or mounting or securing or anchoring or retaining or coupling the receptacle 40 to the mandible 8 (FIG. 1), or the other damaged or degenerated bone materials or members 8, 80, or the like, and may further include one or more or a number of apertures 44 formed therein for allowing the bone materials of the human or user to be filled or grown or engaged into the openings 15 of the spatial units 11 and/or the spatial body member 10.

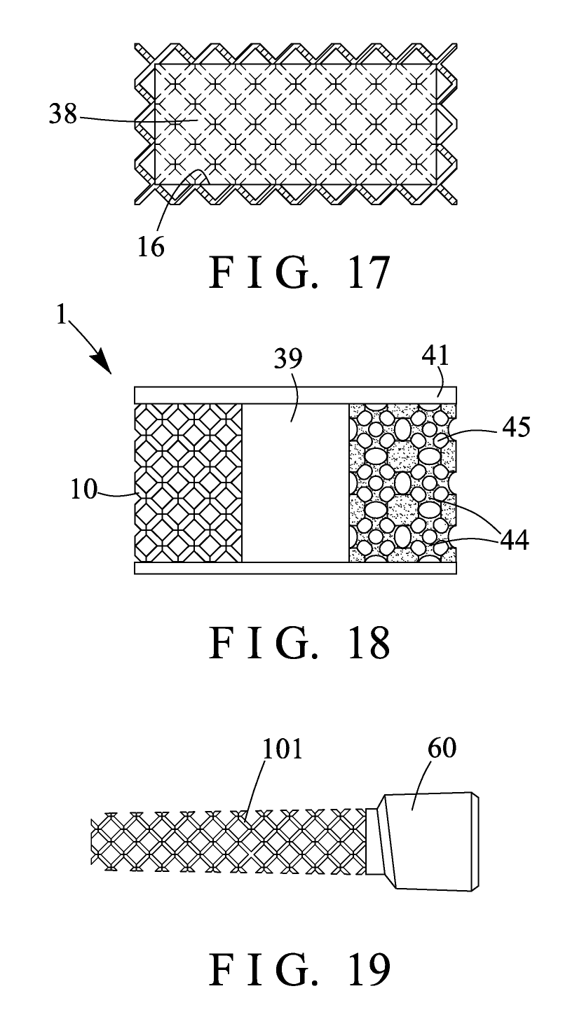

Alternatively, as shown in FIG. 14, the inner core 301 may include a number of cubical or parallelepiped or three dimensional or spatial units 35 having a shape, contour, configuration, structure or the like different from that of the spatial units 11, 21, 31, and preferably includes a compactness or density different from that of the spatial units 11, 21, 31. Alternatively, as shown in FIG. 13, the body member 10 may include a solid core 36 directly disposed or engaged in the chamber 16 thereof, or the other solid cores 37, 38, 39 (FIGS. 15, 17, 18) having a shape, contour, configuration, structure or the like different from that of the solid core 36 as shown in FIG. 13. Further alternatively, as shown in FIG. 18, the textured implant device 1 may include the other cubical or parallelepiped or three dimensional or spatial units 44 disposed or engaged therein for forming or defining the other three dimensional or spatial objects 45.

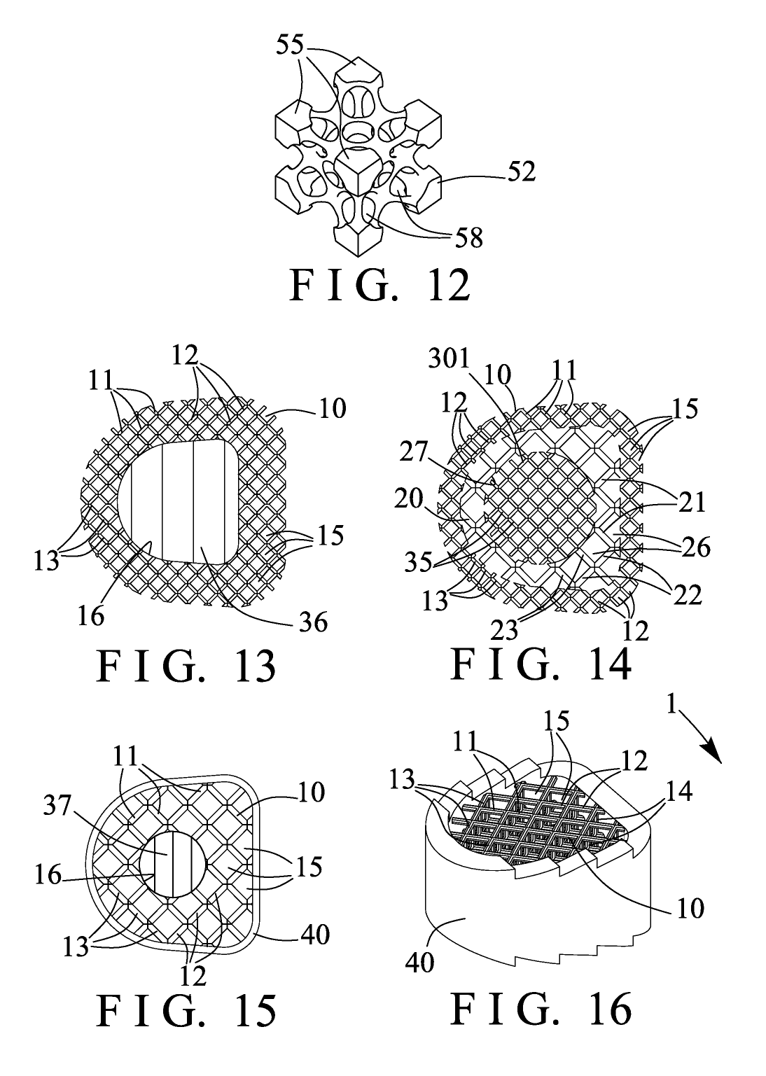

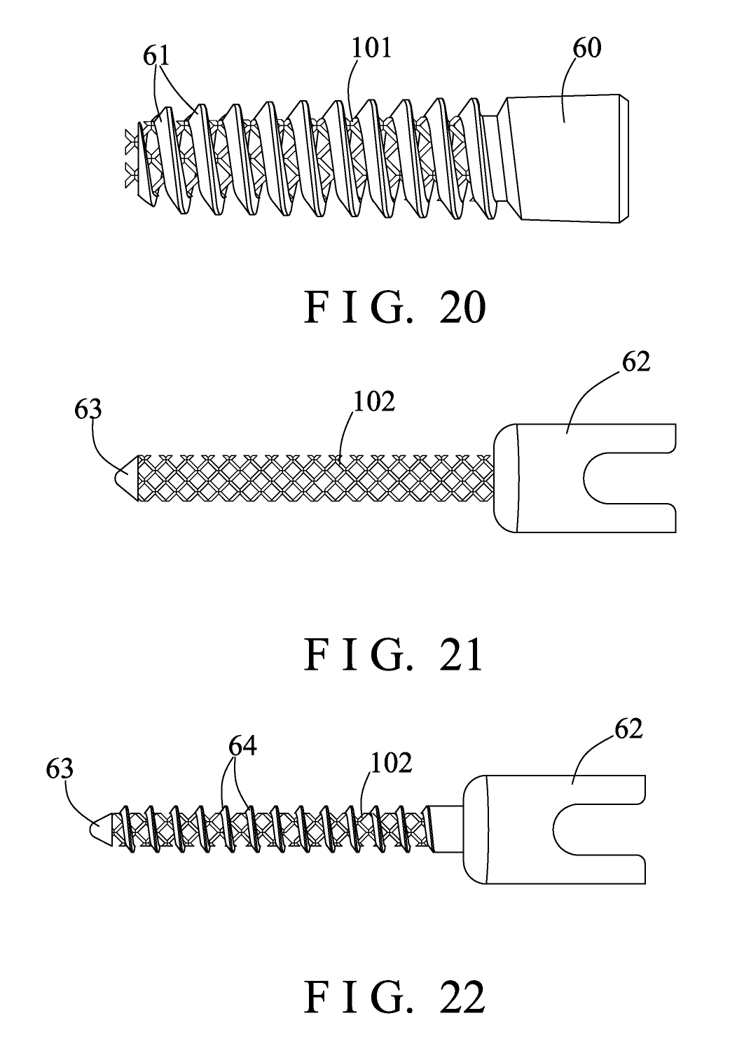

Alternatively, the spatial body member 10, the casing 20, and/or the inner core 30, 301 may include a number of cubical or parallelepiped or three dimensional or spatial units 50, 51, 52, as shown in FIGS. 10-12, different from that of the units 11, 21, 31, 35, 44, and the three dimensional or spatial units 50, 51, 52 each may also include a number of bars 53, 54, 55 attached or mounted or secured or engaged or molded or welded or adhered or secured or coupled together to form or define a number of orifices or cavities or openings 56, 57, 58 in the spatial units 50, 51, 52 respectively. Alternatively, as shown in FIGS. 19-20, the spatial body member 101 may be formed or shaped into the other shape, contour, configuration, structure or the like, and may include a head 60 attached or mounted or secured to one end thereof for forming a bolt or fastener or the like, and may include a helical member 61 attached or mounted or secured thereto, or alternatively, as shown in FIGS. 21-22, the spatial body member 102 may be formed or shaped into the further shape, contour, configuration, structure or the like, such as the screw, bolt, fastener, nail, rod, pole, plate, disc, cage, or block or the like, and may include a head 62 attached or mounted or secured to one end thereof for forming another bolt or fastener or the like, and may include a tip 63 secured thereto, and may include a helical member 64 attached or mounted or secured thereto for forming the bolt or fastener or the like.

In operation, as shown in FIG. 1, the ears or panels or flaps 42, 43 of the receptacle 40 may be used to be solidly and stably attached or mounted or secured or anchored or retained or coupled to the mandible 8, or the other damaged or degenerated bone materials or members 8, or the like; or, as shown in FIG. 7, the spatial body member 10 may also be directly engaged into the damaged or degenerated portion of the mandible 8, or may be disposed or attached or mounted or secured or engaged into the space between two vertebral bodies 81, 82 for forming or acting as the intervertebral disk and for sustain the vertebral bodies 81, 82 in place. The apertures 44 of the receptacle 40 or the openings 15 formed in the spatial units 11, 21, 31, 35, 44 allow the bone materials of the human or user to be filled or grown or engaged into the openings 15 of the spatial units 11, 21, 31, 35, 44, and/or the spatial body member 10.

The spatial units 11, 21, 31, 35, 44 of the spatial body members 10, 20, 30, 45 may be made or manufactured or formed with various kinds of materials, such as the metal, china or porcelain materials, and may be selected form stainless steel, titanium, gold, silver, stellite, oxidized cobalt, tantalum, or the like, or may be made or manufactured or formed with various high molecular components, high polymer materials, or the like, and may be selected form polyetherether ketone (PEEK), polyetherketoneketone (PEKK), ultra-high-molecular-weightpolyethylene (UHMWPE), polylactide (PLA), polyglycolicacid (PGA), polylacticcoglycolicacid (PLGA), or the like, and the bars 12-14, 22-25, 32, 53-55 and the openings 15, 26, 56-58 of the spatial body members 10, 20, 30, 45 may form a resilient or flexible or compressible or deformable structure for resiliently or flexibly or suitably engaged with the damaged or degenerated bone materials or members 8, 80, and for repairing bone defects.

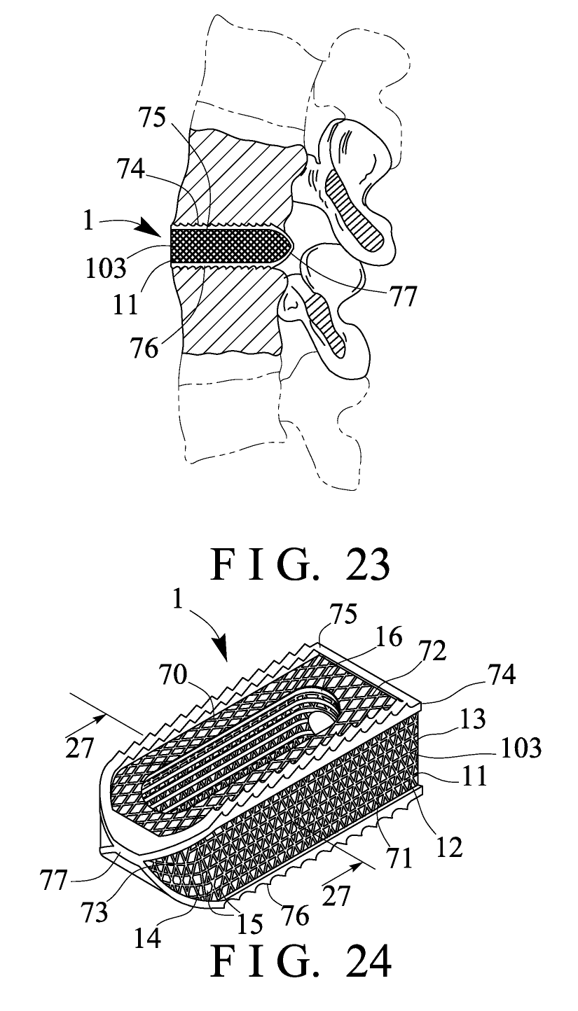

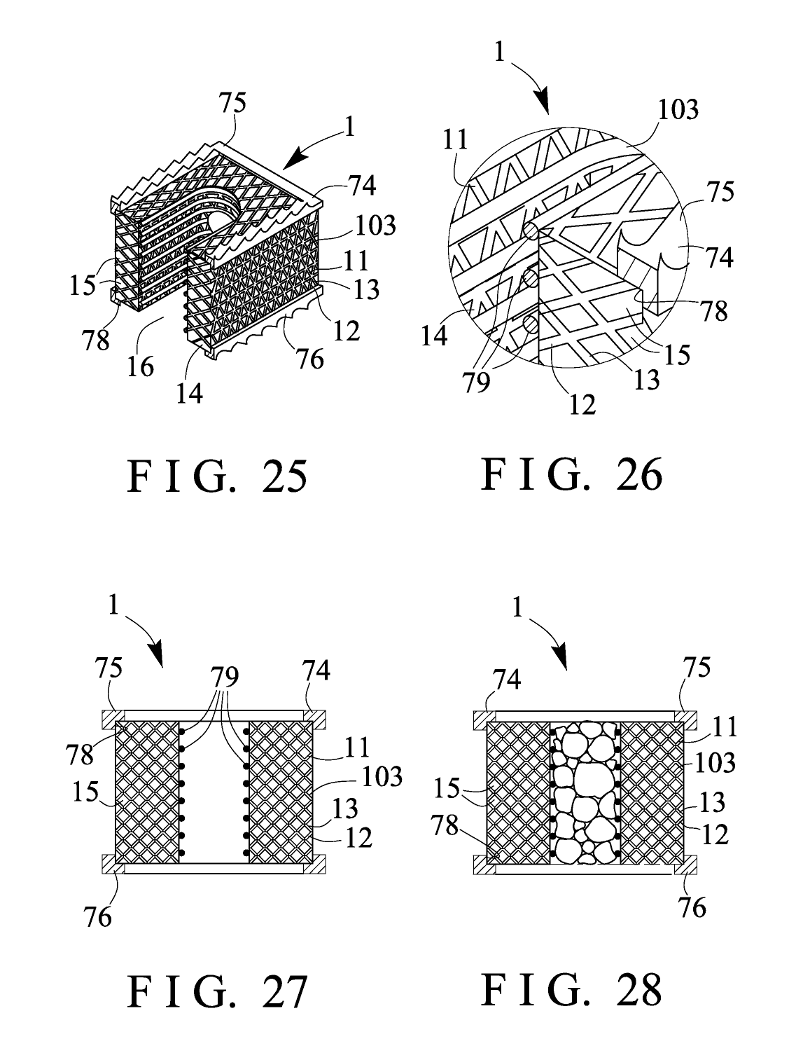

Alternatively, as shown in FIGS. 23-28, the textured implant device 1 also comprises a constructed or textured three dimensional or spatial body member 103 including a porous or perforated structure formed and defined by a number of cubical or parallelepiped or three dimensional or spatial units 11 (FIG. 8) each of which include a number of longitudinal bars 12, a number of lateral bars 13, and a number of vertical bars 14 for forming the cubical or parallelepiped or three dimensional or spatial structure and for forming or defining a number of orifices or cavities or openings 15 in the spatial units 11 and/or in the spatial body member 103, and the longitudinal bars 12 and the lateral bars 13 and the vertical bars 14 of the spatial units 11 may be molded or welded or adhered or secured or coupled together to form the three dimensional or spatial body member 103.

For reinforcing purposes, the spatial body member 103 further includes a chamber 16 formed therein and formed or defined by two side wall panels 70, 71, a rear wall panel 72, and a front wall panel 73, and an outer peripheral housing or casing or receptacle or frame device 74 having a substantially rectangular upper frame member 75 disposed or attached or mounted or secured or engaged with or onto top of the spatial body member 103, and a substantially rectangular lower frame member 76 disposed or attached or mounted or secured or engaged with or onto bottom of the spatial body member 103, in which the front portions 77 of the upper and the lower frame members 75, 76 are welded or secured or coupled together to form a relatively reduced or narrowed or protruded or dimension decreased front portions 77 for the spatial body member 103 and for allowing the textured implant device 1 to be easily and quickly disposed or engaged into the damaged or degenerated spinal column or vertebrae or spinal disc of the human or user.

The upper and the lower frame members 75, 76 of the frame device 74 each include an inner peripheral shoulder or depression or recess 78 formed therein for snuggly fitting or receiving or engaging with the spatial body member 103 and for allowing the upper and the lower frame members 75, 76 of the frame device 74 to be solidly and stably and firmly anchored or retained or positioned or attached or mounted or secured onto the spatial body member 103. The spatial body member 103 may further include a number of peripheral cables or wires or rods or ropes 79 disposed or attached or mounted or secured or engaged in the chamber 16 of the spatial body member 103 and attached or mounted or secured to the side wall panels 70, 71 and the rear wall panel 72 and the front wall panel 73 with such as molding or welding or adhering procedures for further reinforcing the spatial body member 103.

Accordingly, the textured implant device in accordance with the present invention includes a porous or perforated structure having a number of orifices or cavities or openings formed therein and defined by a number of cubical or parallelepiped or spatial units for increasing the resilience of the textured implant device and for preventing the bone materials or members of the user from being damaged by the textured implant device and for allowing the bone materials of the user to be grown and filled into the orifices or cavities or openings of the textured implant device.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A textured implant device comprising:
a spatial body member including a plurality of three dimensional units coupled together to form a spatial and three dimensional structure for said spatial body member, said three dimensional units each including a plurality of bars coupled together to form a plurality of openings therein and for forming a porous structure for said spatial body member.

2. The textured implant device as claimed in claim 1, wherein said spatial body member includes a chamber formed therein, and a casing engaged into said chamber of said body member and coupled to said body member.

3. The textured implant device as claimed in claim 2, wherein said casing includes a plurality of three dimensional units coupled together in regular to form a spatial and three dimensional structure for said casing.

4. The textured implant device as claimed in claim 3, wherein said three dimensional units of said casing each include a plurality of bars coupled together to form a plurality of openings therein and to form a porous structure for said casing.

5. The textured implant device as claimed in claim 2, wherein said casing includes a compartment formed therein, and an inner core engaged into said compartment of said casing and coupled to said casing.

6. The textured implant device as claimed in claim 5, wherein said inner core includes a plurality of three dimensional units coupled together to form a spatial and three dimensional structure for said inner core.

7. The textured implant device as claimed in claim 6, wherein said three dimensional units of said inner core each include a plurality of bars coupled together to form a plurality of spaces therein and to form a porous structure for said inner core.

8. The textured implant device as claimed in claim 5, wherein said inner core includes at least one insert engaged therein.

9. The textured implant device as claimed in claim 1 further comprising a receptacle engaged onto said body member.

10. The textured implant device as claimed in claim 9, wherein said receptacle includes at least one flap extended outwardly therefrom.

11. The textured implant device as claimed in claim 9, wherein said receptacle includes a plurality of apertures formed therein.

12. The textured implant device as claimed in claim 1, wherein said spatial body member is made of a material selected form stainless steel, titanium, gold, silver, stellite, oxidized cobalt, tantalum, polyetherether ketone (PEEK), polyetherketoneketone (PEKK), ultra-high-molecular-weightpolyethylene (UHMWPE), polylactide (PLA), polyglycolicacid (PGA), or polylacticcoglycolicacid (PLGA).

13. The textured implant device as claimed in claim 1, wherein said spatial body member includes a shape selected from a screw, bolt, fastener, nail, rod, pole, plate, disc, cage, or block.

14. The textured implant device as claimed in claim 1, wherein said three dimensional unit is selected from a triangle, rectangle, pentagon, hexagonal, octangle, and the like.

15. A textured implant device comprising:
a spatial body member including a chamber formed therein and formed and defined by two side wall panels, a rear wall panel, and a front wall panel, and
a frame device including an upper frame member attached onto top of said spatial body member, and a lower frame member attached onto bottom of said spatial body member, said upper and said lower frame members having a front portion secured together.

16. The textured implant device as claimed in claim 15, wherein said upper and said lower frame members of said frame device each include an inner peripheral recess formed therein for engaging with said spatial body member.

17. The textured implant device as claimed in claim 15, wherein said spatial body member includes a plurality of peripheral ropes engaged in said chamber of said spatial body member and attached to said side wall panels and said rear wall panel and said front wall panel of said spatial body member.

18. The textured implant device as claimed in claim 15, wherein said upper and said lower frame members of said frame device each include a rectangular shape.

19. The textured implant device as claimed in claim 15, wherein said spatial body member includes a plurality of three dimensional units coupled together to form a spatial and three dimensional structure for said spatial body member, said three dimensional units each include a plurality of bars coupled together to form a plurality of openings therein and for forming a porous structure for said spatial body member.
